(19) United States
(12) Patent Application Publication
(10) Pub. No.: US 2025/0289940 A1
(43) Pub. Date: Sep. 18, 2025

(54) PRETREATMENT OF POLYOLEFIN WASTE TO IMPROVE DEPOLYMERIZATION

(71) Applicant: Basell Poliolefine Italia S.r.l., Milano (IT)

(72) Inventors: Daniel F. White, Houston, TX (US); Sandor Nagy, Seabrook, TX (US); Christopher D. Smith, Kingwood, TX (US); Francesco Menichelli, Milano (IT)

(73) Assignee: Basell Poliolefine Italia S.r.l., Milano (IT)

(21) Appl. No.: 19/076,438

(22) Filed: Mar. 11, 2025

Related U.S. Application Data
(60) Provisional application No. 63/564,237, filed on Mar. 12, 2024.

(51) Int. Cl. C08J 11/12; C07C 4/04; C08J 11/16

(52) U.S. Cl. CPC C08J 11/12; C07C 4/04; C08J 11/16; C08J 2323/06; C08J 2323/12

(57) ABSTRACT

A method for treating a polymer recyclate to produce an improved depolymerization feed is described. The method comprises providing a polymer recyclate comprising a polyolefin component and a polar polymer component, wherein the polar polymer component comprises heteroatom bonds. The polymer recyclate is subjected to reaction conditions sufficient to degrade at least a portion of heteroatom bonds to produce a degraded polar polymer component and a waste gas comprising hetero-containing compounds. An improved depolymerization feed, comprising the polyolefin component and the degraded polar polymer component, is recovered. Also, disclosed is a depolymerization process comprising a step of pretreating a polymer recyclate according to the disclosed method.

### CROSS-REFERENCE TO RELATED APPLICATIONS

The application claims the benefit of priority to U.S. Provisional Patent Application No. 63/564,237 filed on Mar. 12, 2024, which is incorporated herein by reference in its entirety.

### FIELD OF THE INVENTION

The disclosure relates to a method for pretreating polyolefin-based polymer recyclates to produce depolymerization feedstock having a reduced content of catalyst poisons. The disclosure further relates to a process for depolymerizing polyolefin-based polymer recyclates to form useful petrochemical products, such as olefin monomers, wherein the process comprises a pretreatment step to reduce catalyst poisons in the feed to a depolymerization reaction zone.

### BACKGROUND OF THE INVENTION

Heightened standards of living and increased urbanization have led to an increased demand for polymer products, particularly polyolefin plastics. Polyolefins have been frequently used in commercial plastics applications because of their outstanding performance and cost characteristics. Polyethylene (PE), for example, has become one of the most widely used and recognized polyolefins because it is strong, extremely tough, and very durable. This allows for it to be highly engineered for a variety of applications. Similarly, polypropylene (PP) is mechanically rugged yet flexible, is heat resistant, and is resistant to many chemical solvents like bases and acids. Thus, it is ideal for various end-use industries, mainly for packaging and labeling, textiles, plastic parts, and reusable containers of various types.

The downside to the demand for polyolefin plastics is the increase in waste. Post-consumer plastic waste typically ends up in landfills, with about 12% being incinerated and about 9% being diverted to recycling. In landfills, most plastics do not degrade quickly, becoming a major source of waste that overburdens the landfill. Incineration is also not an ideal solution to treating the plastic wastes as incineration leads to the formation of carbon dioxide and other greenhouse gas emissions. As such, there has been much interest in developing methods of recycling plastic waste to reduce the burden on landfills while being environmentally friendly.

A drawback to the recycling of plastic wastes is the difficulty in successfully producing commercially usable or desirable products, such as olefin monomers. Plastic waste recycling currently includes washing the material and mechanically reprocessing it; however, the resulting pellets remain contaminated with impurities such as polar polymers, cellulose, and other non-intentional added substances (NIAS). These impurities render the pellets undesirable for many uses based on both performance and appearance. Further, it is difficult to obtain a pure stream of any particular polymer, resulting in a mixed plastic waste stream that may not have the desired properties post-recycling.

Recent advances have focused on converting plastic waste to useable products like fuel sources or commercially important raw materials. Methods of performing pyrolysis of the plastic waste stream followed by catalytic depolymerization have been developed to generate various products: gases, gasoline fractions, kerosene fractions, diesel fractions, and waxes. Thermal or pyrolytic depolymerization of plastic waste back to constituent oils for the purposes of recycling is an energy intensive process requiring temperatures up to 600° C. Various solid catalysts have been demonstrated to lower the temperature required for pyrolysis. U.S. Pat. No. 11,767,408 discloses depolymerizing polyolefins using supported metal oxides. Pub. Appl. US 2021/0061972 discloses depolymerizing plastics using a fluorinated alumina catalyst. PCT Pub. No. WO 2022/122596 discloses depolymerizing polyolefin-based waste material into useful petrochemical products using a composite of at least one zeolite catalyst with one or more solid inorganic co-catalyst(s). WO 2023/088861 discloses depolymerizing plastic waste using a silica supported heteropolyacids catalyst.

Though catalysts allow pyrolysis at lower temperatures than uncatalyzed pyrolysis, these lower temperatures may still be in the range of from 300° C. to 500° C. Such temperatures are still high enough that catalyst poisons present in most plastic waste will quickly degrade catalytic activity, such that continuous addition of fresh catalyst must be made. This high usage coupled with typical requirement of wt % levels rather than ppm levels of catalyst can lead to much higher costs for depolymerizing plastic waste containing significant amounts of non-polyolefinic material. Therefore, it would be desirable to reduce the amount of non-polyolefinic contaminants in polymer recyclates to mitigate poisoning of depolymerization catalysts, thereby reducing catalyst consumption and cost.

Despite the advances made in recycling polyolefins, there is a continued need for the development of a robust process for the conversion of polyolefin-rich waste feeds to useful petrochemical products. Ideally, these processes will overcome ‘poisoning’ of depolymerization catalysts due to other polymers and contaminants that may be present in the waste feed.

### SUMMARY OF THE INVENTION

The present disclosure provides a method for treating a polymer recyclate to produce an improved depolymerization feed having a reduced content of contaminants that have a negative effect on catalytic activity of depolymerization catalysts. The method comprises adding a polymer recyclate to a reaction zone, wherein the polymer recyclate comprises a polyolefin component and a polar polymer component, wherein the polar polymer component comprises one or more heteroatom bonds. The reaction zone is purged with a gas to maintain an oxygen-free atmosphere in the reaction zone. Reaction conditions are implemented in the reaction zone, the reaction conditions comprising a temperature and a time period sufficient to degrade at least a portion of the one or more heteroatom bonds to produce a degraded polar polymer component and a contaminant gas comprising hetero-containing compounds. However, the temperature and the time period are insufficient to decompose the polyolefin component. An improved depolymerization feed is recovered, wherein the improved depolymerization feed comprises the polyolefin component and the degraded polar polymer component.

In another aspect, the disclosure further discloses a process of depolymerizing a polyolefin recyclate. The process comprises pretreating a polymer recyclate comprising a polyolefin component and a polar polymer component, wherein the polar polymer component comprises one or more heteroatom bonds. The pretreatment step comprises subjecting the polymer recyclate to reaction conditions sufficient to degrade at least a portion of the one or more heteroatom bonds to produce a treated polymer recyclate and a waste gas comprising hetero-containing compounds. The treated polymer recyclate, comprising the polyolefin component and a degraded polar polymer component, is recovered and contacted with a catalyst under depolymerization conditions to produce a liquid product comprising one or more olefin monomers.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram of a depolymerization process according to embodiments of the disclosure;

FIG. 2A and FIG. 2B show GC/MS traces of a first polymer recyclate treated at 250° C. and 300° C., respectively, according to embodiments and/or techniques disclosed herein;

FIG. 3A and FIG. 3B show GC/MS traces of a second polymer recyclate treated at 250° C. and 300° C., respectively, according to embodiments and/or techniques disclosed herein;

FIG. 4A and FIG. 4B show GC/MS traces of a third polymer recyclate treated at 250° C. and 300° C., respectively, according to embodiments and/or techniques disclosed herein;

FIG. 5A and FIG. 5B show GC/MS traces of a fourth polymer recyclate treated at 250° C. and 300° C., respectively, according to embodiments and/or techniques disclosed herein;

FIG. 6 is a drawing of a polymer recyclate treatment apparatus used for measuring visbreaking of a polyolefin component of a polymer recyclate pretreated according to embodiments and/or techniques disclosed herein;

FIG. 7A and FIG. 7B show graphical characterization of molecular weight versus time of a polyolefin-based polymer recyclate treated at 180° C. according to embodiments and/or techniques disclosed herein; and

FIG. 8A and FIG. 8B show graphical characterization of molecular weight versus time of a polyolefin-based polymer recyclate treated at 250° C. according to embodiments and/or techniques disclosed herein.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase. It must also be noted that, as used in the specification and the appended claims, the singular forms “a,” “an,” and “the” include plural references unless otherwise specified.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “activated clay” describes clay or clay minerals, including, but not limited to, smectites, vermiculites, Fuller's earth, or a combination thereof, which have been chemically treated with dilute acids (e.g., sulfuric acid) or an alkaline substance (e.g., sodium hydroxide (NaOH), potassium hydroxide (KOH), or ammonium hydroxide (NH₄OH)) to form a treated clay. The treated clay (acidic or alkaline) can be optionally thermally heat treated between 100° C. and 200° C. to improve to improve their absorbency.

As used herein, “char” refers to coke, a carbon-containing solid, that accumulates on the catalyst particles during pyrolysis.

As used herein, “degradation promoter” refers to a composition that is a catalyst for or otherwise facilitates the decomposition of non-polyolefinic components in the treatment process (i.e., process for treating the polyolefin depolymerization feed containing non-polymeric contaminants). For example, calcium carbonate catalyzes the hydrolysis of ester bonds in certain polymers such as, but not limited to, polyethylene terephthalate.

As used herein, “feed stream” refers to a supply of polyolefin-based material for depolymerization. Depending on the depolymerization unit, the feed stream can be a continuous supply of material or a batch of material. The feed stream can be pure polyolefins or can be a mix of polyolefins with non-polyolefin components.

As used herein, “macroscopically homogeneous mixture,” with respect to a mixture of a polyolefin component and a polar polymer component, means a mixture in which the composition is uniform throughout the mixture. That is to say, that a macroscopically homogeneous mixture of polyolefin component and a polar polymer component contains a dispersed phase of the polar polymer in a matrix phase of the polyolefin component due to incompatibility of the polar polymer component and the polyolefin component. Although this incompatibility prevents a microscopically homogeneous mixture as would be the case with a blend of a HDPE with a LLDPE, the mixture is homogeneous on a macroscopic scale in that random samples of the polymer recyclate would have the same or substantially the same amount of, dispersion of, and average domain size of the polar polymer component in the matrix phase of the polyolefin component regardless of the size the samples or location from which such samples are taken in a bulk amount of the polymer recyclate.

As used herein, “non-polyolefin components” refers to material present in a polyolefin-based feed, or waste, stream that can reduce the abilities of a zeolite and other depolymerization catalysts to catalyze the depolymerization of the polyolefins that are present in the stream. Examples of non-polyolefin components include non-polyolefinic polymers with high oxygen and/or nitrogen content.

As used herein, “poison mitigation compound” refers to a composition that reacts materials in the treatment process (i.e., process for treating the polyolefin depolymerization feed containing non-polymeric contaminants) as a degradation promoter and/or a scavenging compound.

As used herein, “post-consumer waste” refers to a type of waste produced by the end consumer of a material stream.

As used herein, “post-industrial waste” refers to a type of waste produced during the production process of a product.

As used herein, “pretreatment” and “treatment” with respect to polymer recyclates are interchangeable.

As used herein, “reaction zone” refers to a chamber sufficiently enclosed to maintain selected operating conditions within the chamber to produce a desired reaction, such as a pyrolysis reaction zone or a condensing reaction zone. In some embodiments, each reaction zone can be a separate reactor. In some embodiments, a single vessel can contain a plurality of reaction zones.

As used herein, “residence time” refers to the time needed to depolymerize a batch of polymer waste in a depolymerization unit.

As used herein, “scavenging compound” refers to a composition that reacts with the decomposition products of non-polyolefinic components in the depolymerization feed stream to keep such decomposition products from acting as poisons to zeolites in a downstream depolymerization process. For example, calcium carbonate acts as a scavenging agent for carboxylic acids generated in the treatment process (i.e., process for treating the polyolefin depolymerization feed containing non-polymeric contaminants).

As used herein, “thermolysis” refers to a thermal depolymerization reaction occurring in the absence of oxygen.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

As used herein, the terms “depolymerization half time” or “half time of depolymerization” refer to the time needed to achieve a 50% loss of mass of a sample at a specific temperature during a TGA thermolysis reactions. The depolymerization half time is related to the residence time that would be needed for large scale industrial depolymerization reactors.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the disclosure.

The term “pure” as used in reference to the feed stream refers to a feed that is 100% polyolefin, but does not mean that the feed contains only one type of polyolefin. Rather, a “pure” feed stream can have a mixture of polyolefins such as low-density polyethylene, high density polyethylene, polypropylene and combinations thereof.

The terms “polyolefin-based” and “polyolefin-rich”, in reference to materials, feed streams, or waste streams, are used interchangeable to refer to a mixture that is at least 80% polyolefin.

All concentrations herein are by weight percent (“wt. %”) unless otherwise specified.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the invention.

The phrase “substantially all of” means greater than or equal to 95 wt %, greater than or equal to 99 wt %, greater than or equal to 99.5 wt %, or greater than or equal to 99.9 wt %.

The following abbreviations are used herein:

| ABBREVIATION | TERM |
|---|---|
| Beta | Beta (or BEA) zeolite |
| EVA | ethylene vinyl acetate |
| EVOH | ethylene vinyl alcohol |
| HDPE | High density polyethylene |
| LDPE | Low-density polyethylene |
| PE | polyethylene |
| PET | poly(ethylene terephthalate) |
| PP | polypropylene |
| sccm | standard cubic centimeter per minute @ |
| | 0° C./32° F. |
| TGA | Thermogravimetric Analysis |
| wt % | weight percent |
| ZSM-5 | Zeolite Socony Mobil-5 |

### Polymer Recyclate

Polymer recyclates herein have polyolefin component and a polar polymer component. The polar polymer component comprises one or more low polarity polymers, one or more high polarity polymers, or a combination thereof. In some embodiments, low polarity polymers are essentially polyolefins have pendant functional groups containing one or more hetero atoms but are miscible with similar polyolefins. In some embodiments, high polarity polymers have a higher concentration of pendant functional groups and/or hetero bonds on the polymer backbone and are not miscible with any polyolefins. In some embodiments, a polymer recyclate contains dissimilar polyolefins which are incompatible with one another (e.g., polyethylene and polypropylene). In spite of advances in sorting and/or cleaning plastic waste, polymer recyclates still contain impurities and/or incompatibilities limiting their direct use in new products due to poor mechanical and/or optical properties. Therefore, it is desirable to depolymerize polymer recyclates into product streams from which olefinic monomers can be recovered to produce new polymers.

However, even small amounts of polar polymers comprising heteroatoms in the polymer backbone and/or pendant functional groups can be deleterious to catalytic activity of depolymerization catalysts. This can result in failure to achieve the desired product from the depolymerization reaction, increases in cost due to the need for higher catalyst addition rates and a corresponding increase in the amount of spent catalyst waste to be processed, or a combination thereof. These negative effects on the depolymerization process could be mitigated if a polymer recyclate could be pretreated to reduce the hetero atom content of the polymer recyclate to produce an improved depolymerization feedstock. Such pretreatment would also be useful for thermal pyrolysis without a catalyst, since reducing hetero atoms in the depolymerization feed would reduce undesirable hetero atoms in the depolymerization product. It would further be desirable is such pretreatment could also start the depolymerization process by some level of visbreaking of polymer chains.

### Polyolefins

The polyolefin component of a polymer recyclate comprises any polymer derived from C₂-C₂₀ olefin monomers containing only hydrogen and carbon atoms. In some embodiments, the polyolefin component comprises ethylene-based homopolymers and copolymers, propylene-based homopolymers and copolymers, butene-based homopolymers and copolymers, or a combination thereof. In some embodiments, the polyolefin component comprises ethylene-based homopolymers and copolymers, propylene-based homopolymers and copolymers, or a combination thereof. In some embodiments, the polyolefin component comprises ethylene-based homopolymers and copolymers. In some embodiments, the polyolefin component comprises propylene-based homopolymers and copolymers.

In some embodiments, the polyolefin component comprises a low density polyethylene (LDPE), linear low density polyethylene (LLDPE), medium density polyethylene, high density polyethylene (HDPE), and/or polypropylene (PP).

Suitable polyethylenes include ethylene homopolymers and copolymers of units derived from ethylene and units derived from one or more of C₃-C₂₀ alpha-olefins or mixtures thereof. In some embodiments, the units derived from the one or more C₃-C₈ alpha-olefin comonomers are present in amounts up to 15 wt. %, based upon the total weight of the copolymer of ethylene. The ethylene homopolymers and copolymers can be produced using either Ziegler Natta catalyst, chromium-based catalyst, or single-site catalyst, e.g., metallocene catalyst. The ethylene homopolymers and copolymers can be produced using a gas phase process, high pressure process, slurry process, or solution process. Ethylene homopolymers and ethylene-C₃-C₈ alpha-olefin copolymers include very low density polyethylene (VLDPE), low density polyethylene (LDPE), linear low density polyethylene (LLDPE), medium density polyethylene (MDPE) and high density polyethylene (HDPE). VLDPE is defined as having a density of 0.860 to 0.910 g/cm³, as measured by ASTM D-1505 “Column Method.” LDPE and LLDPE are defined as having densities in the range of from 0.90 to 0.930 g/cm³. MDPE is defined as having a density of 0.925 to 0.940 g/cm³. HDPE is defined as having a density of at least 0.945 g/cm³, preferably from 0.945 to 0.969 g/cm³. The ethylene homopolymers and copolymers preferably have melt indexes (MIs), as measured by ASTM D 1238, condition 190° C./2.16 kg, from 0.01 to 400 dg/min., preferably, from 0.1 to 200 dg/min., more preferably from 1 to 100 dg/min.

In some embodiments, LDPE is derived from ethylene homopolymers, copolymers of units derived from ethylene and units derived from one or more of C₃-C₁₂ alpha-olefins, copolymers of units derived from ethylene and units derived from one or more of alpha mono-olefins comprising polar groups, or mixtures thereof.

In some embodiments, LDPE homopolymers can be produced in a high pressure, free-radical polymerization process, such as in one or more tubular reactors, one or more autoclave reactors, or a combination thereof. Operating conditions for the high-pressure process can include, but are not limited to, a pressure in the range of from 70 MPa to 700 MPa and a temperature in the range of from 150° C. to 500° C. Such homopolymers have a high degree of long-chain branching and a density in the range of from 0.910 g/cm³ to 0.940 g/cm³.

In some embodiments, LDPE copolymers of ethylene and C₃-C₁₂ alpha-olefins can be produced in a high pressure, free-radical polymerization process, such as in one or more tubular reactors, one or more autoclave reactors, or a combination thereof. Such C₃-C₁₂ alpha-olefins include, but are not limited to, substituted or unsubstituted C₃ to C₁₂ alpha olefins such as propylene, butene, pentene, hexene, heptene, octene, nonene, decene, undecene, dodecane, and isomers thereof. When present, comonomers can be present in amounts up to 15 wt %, 10 wt %, or 5 wt %. Operating conditions for the high-pressure process can include, but are not limited to, a pressure in the range of from 70 MPa to 700 MPa and a temperature in the range of from 150° C. to 500° C. Such homopolymers have a high degree of long-chain branching and a density in the range of from 0.910 g/cm³ to 0.940 g/cm³.

LDPE as described above, can be characterized by having: i) a density in the range of from 0.90 g/cm³ to 0.940 g/cm³ or from 0.915 g/cm³ to 0.935 g/cm³; ii) a melt index (2.16 kg, 190° C.) less than or equal to 5.0 g/10 min., less than or equal to 1.0 g/10 min., less than or equal to 0.5 g/10 min., less than or equal to 0.2 g/10 min., or less than or equal to 0.1 g/10 min.; iii) a molecular weight distribution (M_{w}/Mₙ) greater than 4.0, greater than 8.0, or greater than 15, and/or less than 35, less than 30, or less than 25; iv) a weight average molecular weight (M_{w}) greater than or equal to 100,000 daltons, greater than or equal to 150,000 daltons, greater than or equal to 200,000 daltons, or greater than or equal to 250,000 daltons, and/or less than or equal to 600,000 daltons, less than or equal to 500,000 daltons, less than or equal to 400,000 daltons, or less than or equal to 300,000 daltons; and v) a melt elasticity (“ER”) greater than or equal to 1.0, greater than or equal to 1.4, or greater than or equal to 2.0.

Suitable polypropylenes include propylene homopolymers and copolymers, including plastomers, having of units derived from propylene and units derived one or more of ethylene and C₄-C₂₀ alpha-olefins or mixtures thereof. Preferably, the units derived from one or more of ethylene and C₄₋C₁₀ alpha-olefin comonomers are present in amounts up to 35 wt. %, based upon the total weight of the copolymer of propylene. The propylene homopolymers and copolymers can be produced using either Ziegler Natta or single-site catalysts, e.g., metallocene catalysts. The propylene homopolymers and copolymers can be produced using a gas phase process, slurry process, or solution process. In some embodiments, when the propylene polymer is a copolymer, it preferably contains 2 to 6 wt. %, based upon the total weight of the copolymer, of ethylene derived units as a comonomer.

### Low Polarity Polymer

In some embodiments, a low polarity polymer is a blend of two or more polymers such as, but not limited to, a blend of EVA, ionomers (such as, but not limited to, EAA and EMAA), EMA, ethylene-based copolymers compatible with polyethylene, or a combination thereof.

In some embodiments, LDPE copolymers of ethylene and one or more of alpha mono-olefins comprising polar groups can be produced in a high pressure, free-radical polymerization process, such as in one or more tubular reactors, one or more autoclave reactors, or a combination thereof. Such alpha mono-olefins comprising polar groups include, but are not limited to, methacrylic acids, esters (e.g., acetate esters, such as vinyl acetate), nitriles, and amides, such as acrylic acid, methacrylic acid, cyclohexyl methacrylate, methyl acrylate, acrylonitrile, acrylamide, or mixtures thereof. When present, comonomers can be present in amounts up to 15 wt %, 10 wt %, or 5 wt %. Operating conditions for the high-pressure process can include, but are not limited to, a pressure in the range of from 70 MPa to 700 MPa and a temperature in the range of from 150° C. to 500° C. Such homopolymers have a high degree of long-chain branching and a density in the range of from 0.910 g/cm³ to 0.940 g/cm³.

LDPE as described above, can be characterized by having: i) a density in the range of from 0.90 g/cm³ to 0.940 g/cm³ or from 0.915 g/cm³ to 0.935 g/cm³; ii) a melt index (2.16 kg, 190° C.) less than or equal to 5.0 g/10 min., less than or equal to 1.0 g/10 min., less than or equal to 0.5 g/10 min., less than or equal to 0.2 g/10 min., or less than or equal to 0.1 g/10 min.; iii) a molecular weight distribution (M_{w}/Mₙ) greater than 4.0, greater than 8.0, or greater than 15, and/or less than 35, less than 30, or less than 25; iv) a weight average molecular weight (M_{w}) greater than or equal to 100,000 daltons, greater than or equal to 150,000 daltons, greater than or equal to 200,000 daltons, or greater than or equal to 250,000 daltons, and/or less than or equal to 600,000 daltons, less than or equal to 500,000 daltons, less than or equal to 400,000 daltons, or less than or equal to 300,000 daltons; and v) a melt elasticity (“ER”) greater than or equal to 1.0, greater than or equal to 1.4, or greater than or equal to 2.0.

### High Polarity Polymer

In some embodiments, a high polarity polymer herein comprises a polymer having an oxygen vapor transmission rate (OVTR), as measured by ASTM D3895, of less or equal to 200 cc·μm/m²·day·atm, less than or equal to 150 cc·μm/m²·day·atm, less than or equal to 100 cc·μm/m²·day·atm, less than or equal to 50 cc·μm/m²·day·atm, or less than or equal to 2 cc·μm/m²·day·atm. A barrier layer can include a cellulose, a polyester, a polyamide, a polycarbonate, a polyvinyl alcohol, a polyurethane, an ethylene vinyl alcohol, or a combination thereof.

Cellulose is a linear polysaccharide made up of β-D-glucopyranose units linked by β(1→4) glycosidic bonds. Each glucose unit in cellulose has three hydroxyl (—OH) groups, which are responsible for the polymer's polar nature. These hydroxyl groups can engage in hydrogen bonding, both within and between cellulose chains. The ability to form multiple hydrogen bonds gives cellulose its crystalline nature and high tensile strength in its natural fibrous form. In the context of recycled plastics, the cellulose that acts as a contaminant typically originates from paper and cardboard materials that are inadvertently mixed with plastic wastes.

Polyesters are a category of polymers that contain the ester functional group in their main chain. While polyethylene terephthalate (PET) is the most common type of polyester used in packaging, several other polyesters also possess properties that can be useful in barrier layers in multilayer films or molded containers. In some embodiments, a polyester comprises polyethylene terephthalate (PET), polyethylene naphthalate (PEN), polybutylene terephthalate (PBT), polycyclohexylenedimethylene terephthalate (PCT), polytrimethylene terephthalate (PTT), polyglycolic acid (PGA), polylactic acid (PLA), polyethylene furanoate (PEF), or a combination thereof.

Polyamides (PAs), often known as nylons, are a group of polymers that provide a good barrier against gases like oxygen and carbon dioxide, making them valuable in packaging, especially for food products that need protection from oxygen to ensure freshness and quality. They also have excellent mechanical properties, chemical resistance, and are typically used in combination with other materials in multilayer films to exploit these properties. In some embodiments, a polyester comprises PA 6 (nylon 6), PA 66 (nylon 6,6), PA 11, PA 12, PA 6/66 copolymer, PA 6/12 copolymer, PA 6/66/6T copolymer, PA 61/6T, MXD6 (Polyamide MXD6), bio-based polyamides, or a combination thereof.

Polycarbonates (PC) are a group of thermoplastic polymers known for their transparency, toughness, and excellent impact resistance. They also have good temperature resistance, making them suitable for applications requiring these properties. However, standard polycarbonate materials generally do not have exceptional barrier properties against gases like oxygen or carbon dioxide, which are often crucial for packaging applications. Still, they are used in multilayer structures for their other properties, often in combination with materials that do provide good gas barrier properties. The term “polycarbonates” refers to a category of polymers that are characterized by the carbonate group in their chemical structures. The most common type of polycarbonate is based on bisphenol A (BPA), but there are several other polycarbonates, including those developed to address concerns about the potential health effects of BPA. In some embodiments, a polycarbonate comprises bisphenol A polycarbonate (BPA-PC), bisphenol S polycarbonate (BPS-PC), bisphenol F polycarbonate (BPF-PC), bisphenol C polycarbonate (BPC-PC), bisphenol Z polycarbonate (BPZ-PC), bisphenol-free polycarbonates, polycarbonate copolymers, aliphatic polycarbonates, or a combination thereof.

Polyvinyl alcohols (PVAs) are synthetic resins prepared by the polymerization of vinyl acetate, followed by hydrolysis of the polyvinyl acetate. They are used in a variety of applications due to their emulsifying and adhesive properties, as well as resistance to oil, grease, and solvents. They are also notable for their barrier properties, particularly in preventing gas and aroma permeabilities, making them useful as a barrier layer in multilayer films. In some embodiments, a polycarbonate comprises fully hydrolyzed PVA, partially hydrolyzed PVA, high molecular weight PVA, low molecular weight PVA, modified PVA, copolymerized PVA, or a combination thereof.

Polyurethanes (PUs) are versatile polymers that are known more for their elasticity, toughness, and chemical resistance than for their barrier properties. However, they can still play a crucial role in certain barrier applications, particularly when resistance to solvents, oils, or greases is required, or when their elasticity and durability provide added value. In some embodiments, a polycarbonate comprises thermoplastic polyurethane (TPU), polyether polyurethane, polyester polyurethane, polycaprolactone polyurethane, aliphatic polyurethane, aromatic polyurethane, waterborne polyurethane, polyurethane foams, bio-based polyurethane, or a combination thereof.

Ethylene vinyl alcohol (EVOH) is a class of polymers known for its exceptional gas barrier properties, high resistance to oils and organic solvents, and flexibility. It is a copolymer of ethylene and vinyl alcohol. EVOH is typically coextruded with other materials in multilayer packaging for food, medical, pharmaceutical, cosmetic, and automotive fuel applications due to its ability to retain gases and flavors and prevent external contamination. In some embodiments, an ethylene vinyl alcohol comprises low ethylene content EVOH (typically around 24% to 32%), medium ethylene content EVOH (typically around 38% to 44%), high ethylene content EVOH (typically around 48% to 72%), or a combination thereof. Higher ethylene content provides more flexibility and better moisture resistance, while higher vinyl alcohol content provides more rigidity and better gas barrier properties.

### Polymer Recyclate Treatment Process

The polymer recyclate added to a reaction zone where it is subjected to reaction conditions comprising a temperature and a time period sufficient to degrade at least a portion of the one or more heteroatom bonds to produce a degraded polar polymer component and a contaminant gas comprising hetero-containing compounds. However, the temperature and the time period are insufficient to decompose the polyolefin component.

An oxygen-free atmosphere is maintained in the reaction zone by purging the reaction zone with a gas. In some embodiments, the purge gas is an inert gas, a light hydrocarbon, or a combination thereof. In some embodiments, the purge gas is nitrogen.

The contaminant gas is generated at a first rate (R1). Purging the reaction zone comprises adding an oxygen-free purge gas to the reaction zone at a second rate (R2). Waste gas, comprising the purge gas and the contaminant gas is withdrawn at a third rate (R3). Therefore, R1+R2=R3. In some embodiments, R2/R1 is greater than or equal to 1, greater than or equal to 2, greater than or equal to 5, greater than or equal to 10.

In some embodiments, reaction conditions further comprise a temperature in the range of from 210° C. to 340° C., from 220° C. to 330° C., from 230° C. to 320° C., from 240° C. to 310° C., or from 250° C. to 300° C.

In some embodiments, reaction conditions further comprise a pressure in the range of from −100 kPag to 70 MPag or from −100 kPag to atmospheric pressure.

In some embodiments, reaction conditions further comprise agitation ranging from stirring to mechanical shear to add specific energy of up to 0.6 kW-hr/kg to the polymer recyclate.

In some embodiments, reaction conditions further comprise a reaction time in the range of from 1 minute to 20 hours.

In some embodiments, the reaction conditions are implemented in an apparatus comprising mixing means, heating means, and/or gas injection and/or withdrawal means. In some embodiments, the apparatus is an extruder.

In some embodiments, the method for treating a polymer recyclate further comprises adding a poison mitigation compound to the polymer recyclate. In some embodiments, the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof. In some embodiments, the poison mitigation compound is added in an amount less than or equal to 20 wt %, from 1 wt % to 10 wt %, or from 2 wt % to 5 wt %, based on the total weight of the polymer recyclate and the poison mitigation compound.

In some embodiments, the reaction conditions are further sufficient to degrade the polyolefin component to form a degraded polyolefin component. In some embodiments, degrading the polyolefin component means visbreaking the polyolefin to reduce the weight average molecular weight (M_{w}), the z-average molecular weight (M_{z}), the intrinsic viscosity (IV), or a combination thereof. In some embodiments, the polyolefin component has a first weight average molecular weight (M_{w1}); the degraded polyolefin component has a second weight average molecular weight (M_{w2}); and M_{w1}/M_{w2} is less than or equal to 0.9, less than or equal to 0.8, less than or equal to 0.7, less than or equal to 0.6, or less than or equal to 0.5.

### Extruder Treatment Method

In some embodiments, the polymer recyclate is fed to an extruder, a mixer, or any apparatus capable of compounding conditions sufficient to melt and mix the polymer recyclate. An oxygen-free atmosphere is maintained in the extruder or mixing apparatus. The compounding conditions will be such that the specific energy from the compounder from shear and/or added heat are sufficient to melt the polymer components and homogenize them with the other components in the mixture in the extruder or mixer. In some embodiments, compounding conditions comprise a temperature in the compounding zone in the range of from 210° C. to 340° C., from 220° C. to 330° C., from 230° C. to 320° C., from 240° C. to 310° C., or from 250° C. to 300° C. In some embodiments, during compounding, the recyclate melt is subjected to a vacuum of up to −100 kPag to further promote evolution of contaminant gas. In some embodiments, an inert gas is injected and subsequently withdrawn to remove contaminant gas from the polymer recyclate melt.

### Depolymerization Process

In some embodiments of the present disclosure, the improved depolymerization feedstock produced by the polymer recyclate treatment method disclosed herein is fed to a depolymerization reaction zone.

In some embodiments, a process for depolymerizing polymers comprises adding the treated polymer recyclate, with or without a depolymerization catalyst to a pyrolysis reaction zone to form a mixture. The mixture is reacted under depolymerization conditions in the absence of oxygen to form a first vapor stream and first liquid stream comprising char. The first vapor stream is added to a first condensation zone, wherein the first vapor product is subjected to condensing conditions to form a second vapor product and a second liquid product comprising one or more olefin monomers.

In further embodiments of the process for depolymerizing polymers, the polyolefin feed stream comprises polyethylene, polypropylene, or a combination thereof. In some embodiments, the polyolefin feed stream comprises up to 20 wt %, up to 15 wt %, up to 10 wt %, or up to 5 wt % of an impurity, wherein the weight percentage is based on the total weight of the polyolefin feed stream. In some embodiments, the impurity comprises one or more members of the group consisting of polyethylene terephthalate, polystyrene, water, chlorine, or a combination thereof.

In some embodiments of the process for depolymerizing polymers, the depolymerization conditions comprise a temperature in the range of from 250° C. to 600° C., from 400° C. to 600° C., from 425° C. to 550° C., or from 450° C. to 500° C., a pressure in the range of from 100 kPa to 1,000 kPa, from 100 kPa to 700 kPa, from 150 kPa) to 600 kPa, or from 200 kPa to 500 kPa, or a combination thereof.

In some embodiments of the process for depolymerizing polymers, the condensing conditions independently comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof.

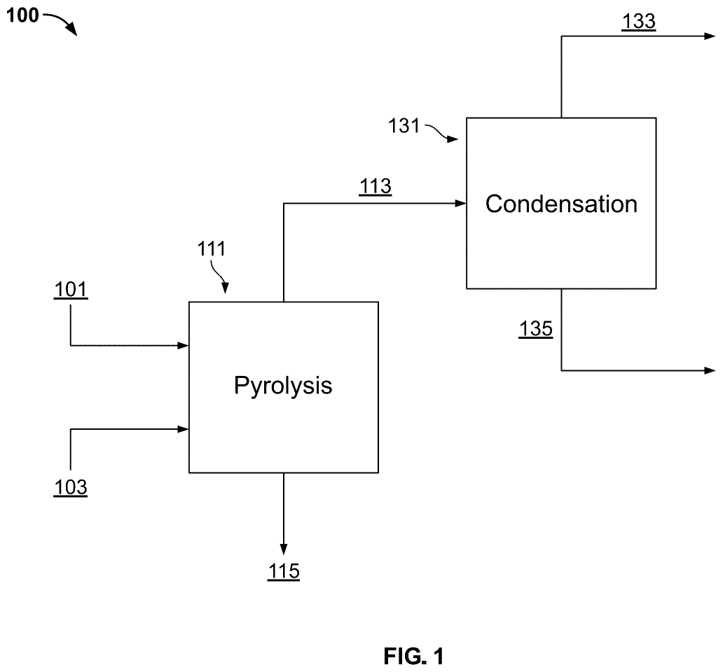

FIG. 1 shows an embodiment of a depolymerization process 100 comprising a pyrolysis reactor 111 and a condensation unit 131. Such embodiment would also include any common equipment associated with distillation columns, including, but not limited to, heat exchangers, pumps, valves, reflux loops, and the like, which are omitted for simplicity.

Polymer recyclate feed 101, a catalyst composition disclosed herein 103, and an inert gas 105, such as nitrogen, are fed to pyrolysis reactor 111. The mixture of catalyst and polymer recyclate are subjected to depolymerization conditions in a reaction zone within pyrolysis reactor 111 to produce a first vapor stream 113 and a liquid stream 115 comprising char.

The vapor stream 113 is fed to condensation unit 131 to be cooled to form a second vapor stream 133 and a second liquid product stream 135 comprising one or more olefin monomers.

### Depolymerization Catalyst

The process in a depolymerization zone is improved by pretreatment of polymer recyclate feed by the method disclosed herein, in that the reduction in non-polyolefinic materials in the polymer recyclate results in a reduced concentration of such contaminants in the polymerization reaction zone. This results in increased catalytic activity (e.g., at the same catalyst addition rate), reduced catalyst addition rate (e.g., to maintain the same catalytic activity, or a combination thereof.

Depolymerization catalysts useful in combination with the pretreatment of polymer recyclate feed by the method disclosed herein include but are not limited to acidic catalysts, metal-based catalysts, thermal catalysts, activated carbon, or combinations thereof. Exemplary acidic catalysts include but are not limited to zeolites (e.g., ZSM-5, Y-zeolite, and beta-zeolite). These are commonly used in catalytic pyrolysis to enhance the production of specific hydrocarbons and limit the formation of waxes and heavy fractions. Exemplary metal-based catalysts include but are not limited to nickel-based catalysts (e.g., Ni/Al₂O₃, Ni/SiO₂), palladium-based catalysts, and other metal-based catalysts. These are used to favor certain depolymerization routes and improve the yield of gases or specific hydrocarbons. Exemplary thermal catalysts include but are not limited to alkali metal hydroxides (e.g., NaOH, KOH). These can promote certain reaction pathways in pyrolysis can also play roles in the pyrolysis process, although their use might be less common. Activated carbon can be used as a catalyst or co-catalyst in pyrolysis due to its ability to promote certain reactions and its high surface area.

Further useful depolymerization catalysts are disclosed in Publ. Pat. Apps. US2023/0106395A1, US2023/0009131A1, US2022/0176358A1, US2022/0135760A1, US2022/0112352A1, US2021/0070958A1, US2021/0070959A1, and US2021/0061972A1.

### Certain Embodiments

Disclosed is a method for treating a polymer recyclate to produce an improved depolymerization feed, the improvement being a reduction in the amount of non-polyolefinic materials present in the polymer recyclate, wherein such non-polyolefinic materials are poisons to the catalysts used in depolymerization processes. A polymer recyclate to be treated by the method herein comprises a polyolefin component and a polar polymer component, wherein the polar polymer component comprises one or more heteroatom bonds. The method comprises adding a polymer recyclate to a reaction zone, wherein the polymer recyclate comprises a polyolefin component, and a polar polymer component, wherein the polar polymer component comprises one or more heteroatom bonds. The reaction zone is purged with a gas to maintain an oxygen-free atmosphere in the reaction zone Reaction conditions are implemented in the reaction zone, the reaction conditions comprising a temperature and a time period sufficient to degrade at least a portion of the one or more heteroatom bonds to produce a degraded polar polymer component and a contaminant gas comprising hetero-containing compounds and insufficient to decompose the polyolefin component. An improved depolymerization feed is recovered, wherein the improved depolymerization feed comprises the polyolefin component and the degraded polar polymer component. In some embodiments, the method is further characterized by one or more of the following:
a) the contaminant gas is generated at a first rate (R1), purging the reaction zone comprises adding an oxygen-free purge gas to the reaction zone at a second rate (R2) and withdrawing a waste gas, comprising the purge gas and the contaminant gas, at a third rate (R3), wherein R1+R2=R3, and R2/R1 is greater than or equal to 1, greater than or equal to 2, greater than or equal to 5, greater than or equal to 10;
b) the polyolefin component comprises polyethylene and/or polypropylene in an amount greater than or equal to 50 wt %, greater than or equal to 60 wt %, greater than or equal to 70 wt %, greater than or equal to 80 wt %, greater than or equal to 90 wt %, or greater than or equal to 95 wt %, based on the weight of the polymer recyclate;
c) the polar polymer component comprises polyvinyl chloride, polyethylene terephthalate, polystyrene, polyamides, polycarbonate, ethylene vinyl alcohol, rubber, or a combination thereof;
d) the reaction conditions further comprise:
   i) a temperature in the range of from 210° C. to 340° C., from 220° C. to 330° C., from 230° C. to 320° C., from 240° C. to 310° C., or from 250° C. to 300° C.;
   ii) a pressure in the range of from −100 kPag to 70 MPag or from −100 kPag to atmospheric pressure;
   iii) agitation ranging from stirring to mechanical shear to add specific energy of up to 0.6 kW-hr/kg to the polymer recyclate;
   iv) a reaction time in the range of from 1 minute to 20 hours; or
   v) a combination thereof;
e) reaction conditions are implemented in an apparatus comprising mixing means, heating means, and/or gas injection and/or withdrawal means, wherein in further embodiments, the apparatus is an extruder;
f) the method further comprising adding a poison mitigation compound to the polymer recyclate, wherein in further embodiments:
   i) the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof, wherein in yet further embodiments, the smectite is a montmorillonite, a bentonite, or a combination thereof;
   ii) the poison mitigation compound is added in an amount less than or equal to 20 wt %, from 1 wt % to 10 wt %, or from 2 wt % to 5 wt %, based on the total weight of the polymer recyclate and the poison mitigation compound; or
   iii) a combination thereof;
g) the reaction conditions are further sufficient to degrade the polyolefin component to form a degraded polyolefin component, wherein in further embodiments:
   i) the polyolefin component has a first weight average molecular weight (M_{w1});
   ii) the degraded polyolefin component has a second weight average molecular weight (M_{w2}); and
   iii) M_{w1}/M_{w2} is less than or equal to 0.9, less than or equal to 0.8, less than or equal to 0.7, less than or equal to 0.6, or less than or equal to 0.5.

In another aspect, a process of depolymerizing a polymer recyclate is disclosed. The process comprises pretreating a polymer recyclate according to any one of the embodiments of the method to produce an improved depolymerization feed as disclosed above. The treated polymer recyclate is then added to a reaction zone in the absence of oxygen under depolymerization conditions sufficient to form a first vapor stream and first liquid stream comprising char. The first vapor stream is added to a condensation zone wherein heat is removed to form a second vapor stream and a second liquid stream comprising one or more olefin monomers. In some embodiments, the method for pretreating the polymer recyclate is performed in an extruder, and the depolymerization if performed in a reactor vessel. In some embodiments, the method for pretreating the polymer recyclate and the depolymerization are performed sequentially in a reactor vessel.

In some embodiments, the process of depolymerizing a polymer recyclate is further characterized by one or more of the following:
a) the process further comprises adding a up to 20 wt % of catalyst to the reaction zone, based on the total weight of the improved depolymerization feed and the catalyst, wherein in further embodiments:
   i) the catalyst comprises an acidic catalyst, a metal-based catalysts, a thermal catalyst, an activated carbon, or a combination thereof;
   ii) the catalyst is present in an amount in the range of from 0.5 wt % to 10 wt %, or from 1 wt % to 5 wt %; or
   iii) a combination thereof.
b) the improved depolymerization feed comprises polyethylene, polypropylene, or a combination thereof;
c) the depolymerization conditions comprise a temperature in the range of from 400° C. to 600° C., a pressure in the range of from 1.0 barg (100 kPa) to 7.0 barg (700 kPa), or a combination thereof; and.
d) conditions in the condensing zone comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof.

The presently disclosed methods of pretreating polymer recyclate as feed to a depolymerization reaction zone are exemplified with respect to the examples below. These examples are included to demonstrate embodiments of the appended claims. However, these are exemplary only, and the invention can be broadly applied to any combination of polyolefin-based feed, with and without non-polyolefin components. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Experimental Method

The experiments herein demonstrate an approach to improve the performance of a chemical recycling operation by reducing catalyst poisons in a polymer recyclate. The polymer recyclate herein comprises a majority of a polyolefin component with the balance being a polar, or non-polyolefinic, polymer component. The polar polymer component comprises one or more heteroatom bonds on the polymer chain, in a functional group pendent from the polymer chain, or a combination thereof. Polyolefinic waste is often contaminated with heteroatomic contaminants such as, but not limited to, cellulose, polyamide (PA), polyethylene terephthalate (PET), etc. The presence of these materials reduces the efficiency of catalysts and results in high level of undesired 0-containing and N-containing compounds in the resulting depolymerization liquids and gases intended for olefin synthesis (e.g., steam cracking or catalytic cracking).

### Polymer Characterization

In Examples 1-13, various polyolefins and non-polyolefinic polymer contaminants were subjected to depolymerization conditions in a lab scale depolymerization unit. The depolymerization unit was a Thermogravimetric Gravimetric Analysis (TGA) instrument. For the TGA thermolysis reactions, the uniform samples were heated under nitrogen at 10° K/min. from a temperature of 100° C. to a hold temperature of 400° C. in a Mettler Toledo™ TGA/DSC 3+ (Mettler Toledo, Columbus, Ohio) and held at 400° C. for 1 hour. After the hold period, each sample was further heated under nitrogen at 10° K/min. from a temperature of 400° C. to a temperature of 600° C. These data are reported in Table 1.

The depolymerization half time (t_{1/2}) at a specific temperature, defined as the time needed to achieve a 50% loss of mass, for various polyolefinic (Examples 10-13) and non-polyolefinic (Examples 1-9) polymers are shown in Table 1 below, Where shown, the depolymerization half time was determined under the assumption of first order decomposition kinetics as t_{1/2}=0.693/k, where k is the first order rate constant determined graphically using a Ln(C₀/C) vs. time plot, wherein C₀ is the initial concentration of a reactant and C is the concentration of the reactant at time t—i.e., t_{1/2} is the time when C₀/C equals 2.

The depolymerization half time is related to the residence time needed in a large scale depolymerization unit. The shorter the half time, the shorter the residence time for a batch of a polymer feed in a depolymerization unit, and the faster the depolymerization rate k. Polyolefins (Examples 10-13) show less loss of mass in the ramp-up from 100° C. to 400° C. than non-polyolefinic polymers (Examples 1-9). In Examples 1-4, more than half (57 wt % to 90 wt %) the mass of the relevant polymers was lost before reaching the 400° F. hold temperature. These data suggested a potential opportunity for separation of polyolefins and non-polyolefinic contaminants in the range of from 100° C. to 400° C.

TABLE 1

| | | Loss, 100° C.- | Loss, 400° C.² | Residue, | k; | t_{1/2} |
|---|---|---|---|---|---|---|
| Ex. | Polymer | 400° C.¹ (wt %) | hold (wt %) | 600° C.³ (wt %) | 400° C. | (min.) |
| 1 | PU (Pellets) | 90% | | 2% | — | — |
| 2 | Cellulose | 89% | | 7% | — | — |
| 3 | PVC | 80% | | 15% | — | — |
| 4 | PU (Foam) | 57% | 46% | 15% | — | — |
| 5 | EVOH | 18% | 95% | 0% | 0.21 | 3.3 |
| 6 | Nylon-6 | 11% | 93% | 4% | — | — |
| 7 | PS | 8% | 100% | 0% | 0.3595 | 1.9 |
| 8 | PET (Bottle) | 2% | 83% | 13% | 0.0516 | 13.4 |
| 9 | Nylon-12 | 1% | 78% | 0% | 0.0243 | 28.5 |
| 10 | PP | 1% | 60% | 1% | 0.014 | 49.5 |
| 11 | HDPE + PP (1:1) | 2% | 35% | 0% | 0.0073 | 94.9 |
| 12 | LDPE | 0% | 25% | 0% | 0.0048 | 144.4 |
| 13 | HDPE | 0% | 20% | 1% | 0.0035 | 198 |

¹Loss during heat-up from 100° C.-400° C.
²Loss during 1 hour hold at 400° C. vs. initial weight at 400° C.
³Residue after continued heat-up to 600° C. vs. starting weight

### Treatment of Polymer Recyclates

The starting materials for Examples 14-21 were four different grades of plastic waste materials (PR1, PR2, PR3, and PR4) obtained from Source One GmbH, Leiferde, Germany. The common characteristic of all of P1-P4 was that they all contained at least 90 wt % polyolefin—polyethylene, polypropylene, or a combination thereof. Candidate materials for the remainder of each of P1-P4 included acrylate polymers, acrylic rubber, cellulose, isoprene polymers, methyl methacrylate polymers, natural rubber, poly(butyl acrylate), polyesters, PVC, vinyl acetate polymers, or mixtures thereof. Actual composition of the “contaminants” (non-polyolefinic polymers) in each of P1-P4 can be inferred from the gases evolved during heating as demonstrated in the examples below.

Frontier Rx-3050TR Tandem micro-Reactor connected to an Agilent GC/MS with a flow of 200 ml/min of helium. Each sample was heated to a specified temperature for 1 minute. Contaminant gas evolving during the heating period was cryo-trapped at the head of the GC/MS column for subsequent separation and identification.

### Determination of Contaminant Gas Composition

The concentration of various hetero-containing gases was measured with headspace Gas Chromatography analysis (GCD) (Pyrolysis-GC analysis using a Rx-3050TR Tandem micro-Reactor, Frontier labs, Fukushima, JP; and an Agilent 8890 GC equipped with a 5977B MSD and UA1 (30m×250 μm×2 μm) GC column, Agilent Technologies, Santa Clara, California.

GC/MS, or gas chromatography-mass spectrometry, was used to identify and quantify hetero-containing compounds in the samples, indicating degradation of polar polymers. This technique is particularly useful for analyzing non-intentionally added substances (NIAS) volatiles, which are chemical compounds that are not intentionally added to a product or material, but can be present as impurities or byproducts of manufacturing processes. In the context of studying the potential effects of reducing hetero-containing compounds, GC-MS can be used to identify specific hetero-containing compounds that are present in a sample and determine their concentrations. This information can be used to compare the contaminant gas recovered from different polymer recyclates and/or the effect of temperature on evolution of such contaminant gas.

Table 3 below provides a list of predominant contaminant gas constituents identified in the examples. Other hetero-containing compounds identified in the examples may indicate that other polar polymers were present in the tested polymer recyclate or may be the result of secondary reactions of compounds released during degradation of the polar polymers.

TABLE 3

| Hetero-containing | Polar polymers that could be indicated by |
|---|---|
| compound in | hetero-containing compound in contaminant |
| contaminant gas | gas |
| Carbon dioxide | polyesters, cellulose, acrylate polymers, maleic |
| | anhydride polymers, and/or polyamides |
| Acetaldehyde | vinyl alcohol polymers, cellulose type polymers, |
| | and/or polyesters |
| Chloromethane | PVC |
| Acetic acid | vinyl acetate polymers |
| Methyl methacrylate | methyl methacrylate polymers |
| Furfural | cellulose |
| Levoglucosenon | cellulose |
| Butanol | poly(butyl acrylate) and/or acrylic rubber |
| Limonene | natural rubber and/or isoprene polymers |
| Levoglucosan | cellulose |
| Caprolactam | polyamides |
| HCl | PVC |
| glucopyranose | cellulose |

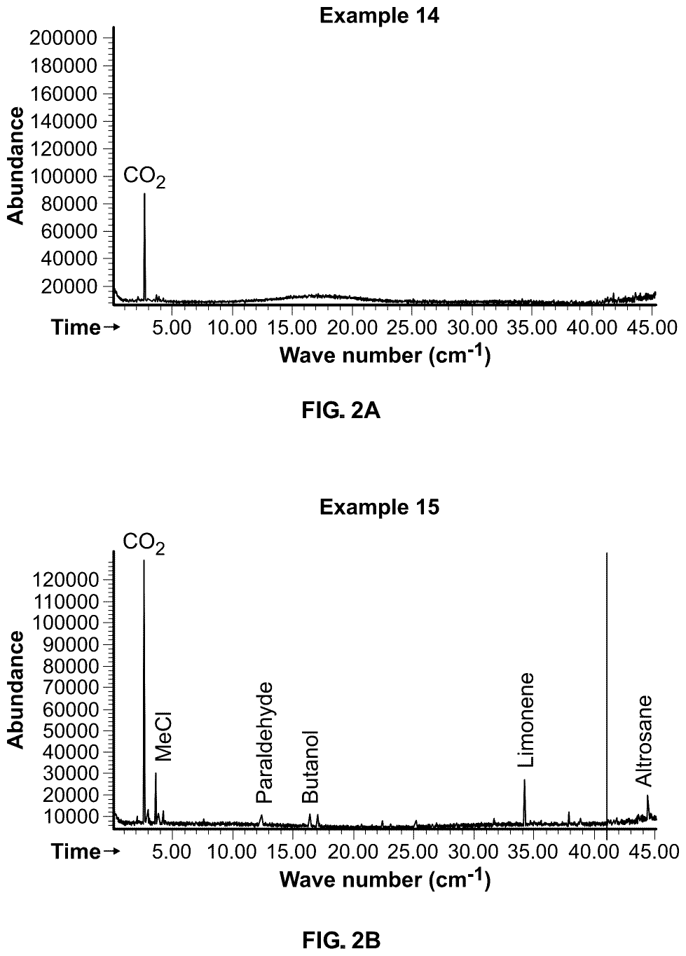

The results of Examples 14-21 are shown in FIG. 2A-FIG. 5B and summarized in Table 4 below. Table 4 further interprets the graphical results in the figures in light of Table 3 to show potential polar polymer constituents of PR1-PR4.

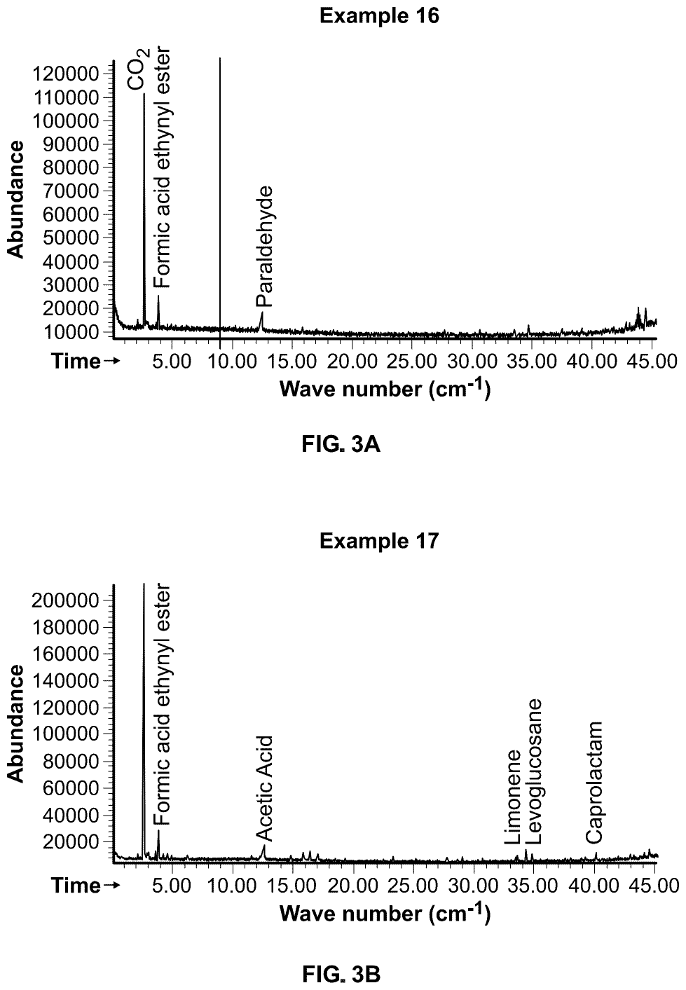

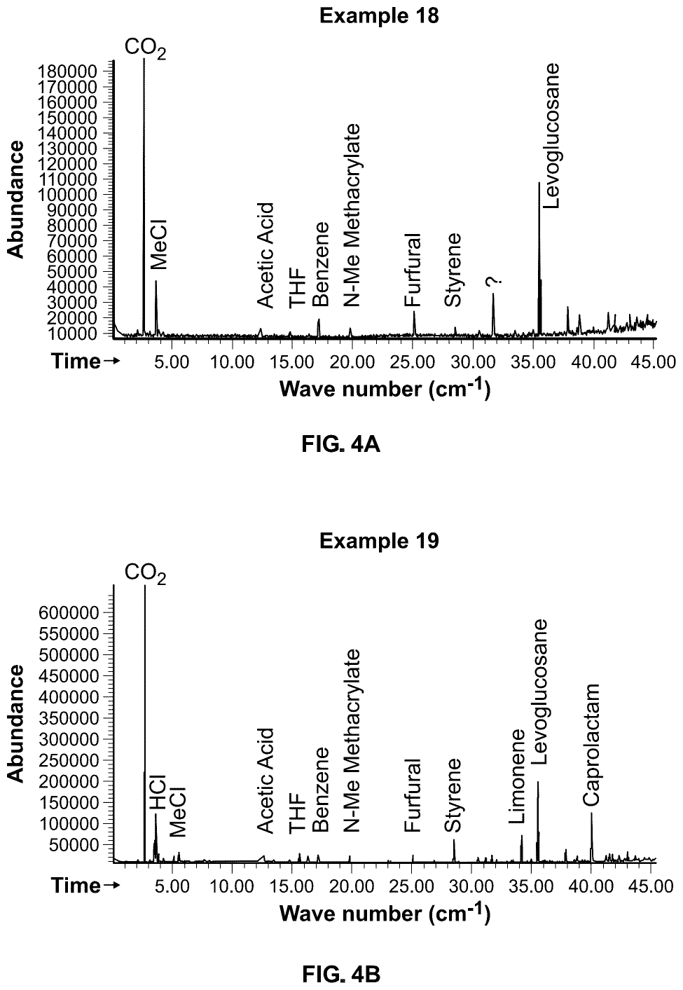

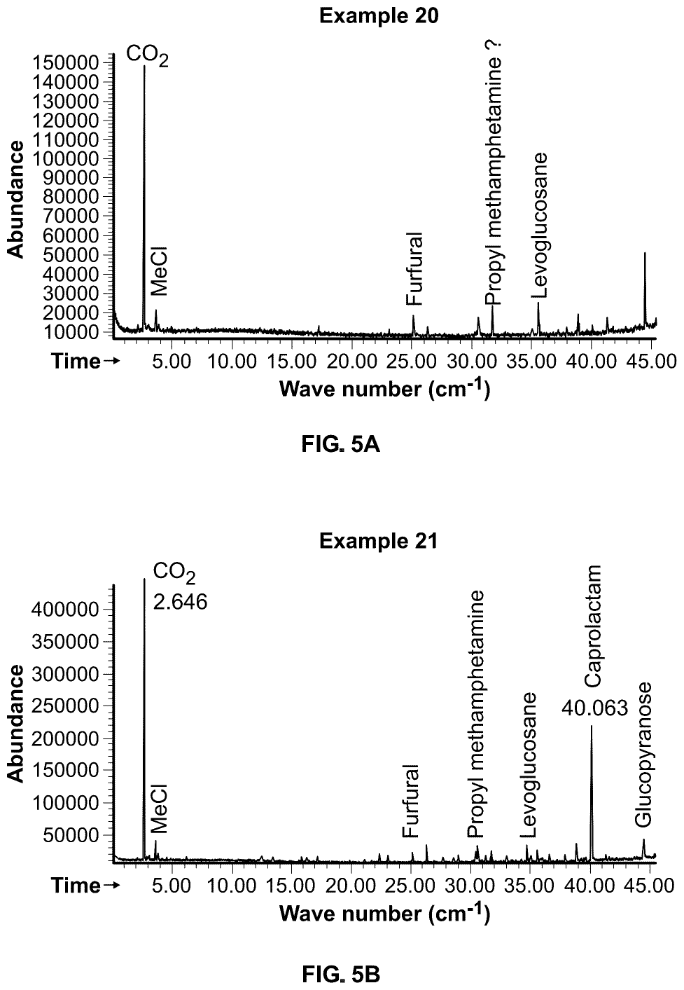

TABLE 4

| | | | GC/MS | |
|---|---|---|---|---|
| | Pol. | Temp. | Trace | |
| Ex. | Rec. | (° C.) | Results | Potential polar polymers degraded |
| 14 | PR1 | 250 | FIG. 2A | polyesters, cellulose, acrylate polymers, maleic anhydride polymers, and/or |
| | | | | polyamides |
| 15 | PR1 | 300 | FIG. 2B | additional degradation of polyesters, cellulose, acrylate polymers, maleic anhydride |
| | | | | polymers, and/or polyamides relative to Example 14 |
| | | | | new degradation of poly(butyl acrylate), acrylic rubber, natural rubber and/or isoprene |
| | | | | polymers relative to Example 14 |
| 16 | PR2 | 250 | FIG. 3A | polyesters, cellulose, acrylate polymers, maleic anhydride polymers, polyamides, |
| | | | | and/or vinyl acetate polymers |
| 17 | PR2 | 300 | FIG. 3B | additional degradation of polyesters, cellulose, acrylate polymers, maleic anhydride |
| | | | | polymers, polyamides, and/or vinyl acetate polymers relative to Example 16 |
| | | | | new degradation of natural rubber and/or isoprene polymers, cellulose, and/or |
| | | | | polyamides relative to Example 16 |
| 18 | PR3 | 250 | FIG. 4A | polyesters, cellulose, acrylate polymers, maleic anhydride polymers, polyamides, |
| | | | | PVC, vinyl acetate polymers, and/or methyl methacrylate polymers |
| 19 | PR3 | 300 | FIG. 4B | additional degradation of polyesters, cellulose, acrylate polymers, maleic anhydride |
| | | | | polymers, polyamides, PVC, vinyl acetate polymers, and/or methyl methacrylate |
| | | | | polymers relative to Example 18 |
| | | | | new degradation of natural rubber, isoprene polymers and/or polyamides relative to |
| | | | | Example 18 |
| 20 | PR4 | 250 | FIG. 5A | polyesters, cellulose, acrylate polymers, maleic anhydride polymers, polyamides, |
| | | | | and/or PVC |
| 21 | PR4 | 300 | FIG. 5B | additional degradation of polyesters, cellulose, acrylate polymers, maleic anhydride |
| | | | | polymers, polyamides, and/or PVC relative to Example 20 |

Examples 14, 16, 18, and 20 show the potential degraded polar polymer constituents for PR1-PR4, respectively, at 250° C. Examples 15, 17, 19, and 21 intensities for hetero-containing compounds released at 250° C. all increased at 300° C. Additionally, Examples 15, 17, 19, and 21 all show the evolution of additional hetero-containing compounds at 300° C. as compared to those generated in Examples 14, 16, 18, and 20 at 250° C. intensities for hetero-containing compound released at 250° C. all increased at 300° C. Furthermore, the potential polar polymer constituents of PR1-PR4 as shown in Table 4 correspond with the candidate polar polymer constituents of PR1-PR4.

### Polyolefin Visbreaking Experimental Method

Additional experiments were performed with PR2 to determine the effects of the pretreatment method on the polyolefin component of the polymer recyclate. Specifically, it was to be determined if any visbreaking of the polyolefin component would occur in conjunction with degradation of the polar polymer component.

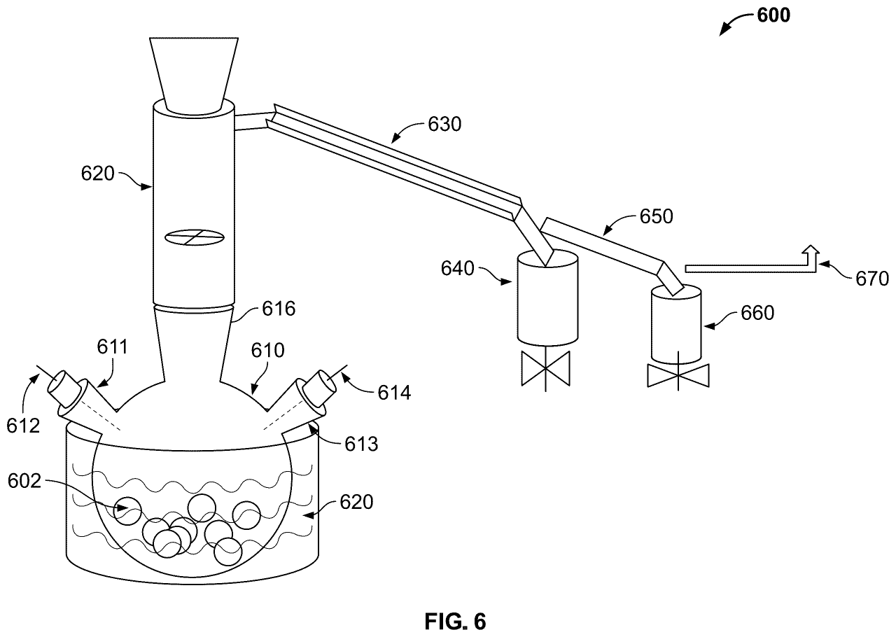

Visbreaking experiments were carried out in a visbreaking apparatus (VBA) 600 as illustrated in FIG. 6. The VBA 600 consists of a 3-neck 500 mL round bottom flask 610 to which a vent column 620, first Liebig condenser 630, first collector tank 640, second Liebig condenser 650, second collector tank 660, and vent 670 are sequentially connected. Vent column 620 is connected to neck 616 in a sealed manner. All items downstream of the vent column 620 are connected to each other in a sealed manner. Pellets of polymer sample 602 are placed in the round bottom flask 610, which is put on an electrical heating mantle 620 to maintain the polymer sample 602 at the target temperature. Nitrogen 612 is injected into a sealed connection to neck 611. Temperature monitor 613 is injected into a sealed connection to neck 613 to monitor the temperature to which the polymer sample 602 is exposed. Those portions of the flask 610 above the heating mantle, in addition to the vent column 620 are wrapped with several layers of aluminum foil to ensure that condensation only occurs in the first Liebig condenser 630 or the second Liebig condenser 650. Condensate from any off-gas generated by the heated polymer sample 602 is collected in the first collector tank 640 and the second collector tank 660. All operations are carried out at atmospheric pressure.

In all experiments, 30 grams of a polymer sample 602 are added to the flask 610 at the beginning of each experiment. Each polymer sample 602 is exposed to a selected temperature for a selected time period. The weight average molecular weight (M_{w}), z-average molecular weight (M_{z}), and intrinsic viscosity of each sample 602 are measured before and after each experiment.

### Examples 22-28

In each of Examples 22-28, approximately 30 g of PR2 polymer recyclate was added to the apparatus shown in FIG. 6. The oil bath 620 is heated to the desired temperature and nitrogen is injected into the apparatus via neck 611 to implement a flow of ______ m³/min. of nitrogen through the headspace above the polymer sample. Each sample was heated to a temperature (180° C. or 250° C.) and for a time period (1 hour, 6 hours, or 16 hours) as shown in Table 5 below. Contaminant gas evolving during the heating period was collected from the nitrogen purge in a cryo-probe and analyzed by an integrated GC/MS instrument.

### Determination of Molecular Weights

Molecular weight distribution (“MWD”) as well as the molecular weight averages (number-average molecular weight, Mₙ weight-average molecular weight, M_{w}, and z-average molecular weight, M_{z}) are determined using a high temperature Polymer Char gel permeation chromatography (“GPC”), also referred to as size exclusion chromatography (“SEC”), equipped with a filter-based infrared detector, IR5, a four-capillary differential bridge viscometer, and a Wyatt 18-angle light scattering detector. Mₙ, M_{w}, M_{z}, MWD, and short chain branching (SCB) profiles are reported using the IR detector, whereas long chain branch parameter, g′, is determined using the combination of viscometer and IR detector at 145° C. Three Agilent PLgel Olexis GPC columns are used at 145° C. for the polymer fractionation based on the hydrodynamic size in 1,2,4-trichlorobenzene (TCB) with 300 ppm antioxidant butylated hydroxytoluene (BHT) as the mobile phase. 16 mg polymer is weighted in a 10 mL vial and sealed for the GPC measurement. The dissolution process is obtained automatically (in 8 ml TCB) at 160° C. for a period of 1 hour with continuous shaking in an Agilent autosampler. 20 μL Heptane was also injected in the vial during the dissolution process as the flow marker. After the dissolution process, 200 μL solution was injected in the GPC column. The GPC columns are calibrated based on twelve monodispersed polystyrene (PS) standards (provided by PSS) ranging from 578 g/mole to 3,510,000 g/mole. The comonomer compositions (or SCB profiles) are reported based on different calibration profiles obtained using a series of relatively narrow polyethylene (polyethylene with 1-hexene and 1-octene comonomer were provided by Polymer Char, and polyethylene with 1-butene were synthesized internally) with known values of CH₃/1000 total carbon, determined by an established solution NMR technique. GPC one software was used to analyze the data. The long chain branch parameter, g′, is determined by the equation:

$${g}^{′} = [ η ] / {[ η ]}_{lin}$$

where, [η] is the average intrinsic viscosity of the polymer that is derived by summation of the slices over the GPC profiles as follows:

$$[ η ] = \frac{\sum {{c}_{i} [ η ]}_{i}}{\sum {c}_{i}}$$

where cᵢ is the concentration of a particular slice obtained from IR detector, and [η]ᵢ is the intrinsic viscosity of the slice measured from the viscometer detector. [η]ₗᵢₙ is obtained from the IR detector using Mark-Houwink equation ([η]ₗᵢₙ=ΣKMᵢ^{alpha}) for a linear high density polyethylene, where Mᵢ is the viscosity-average molecular weight for a reference linear polyethylene, K and α are Mark-Houwink constants for a linear polymer, which are K=0.000374, α=0.7265 for a linear polyethylene and K=0.00041, α=0.6570 for a linear polypropylene.

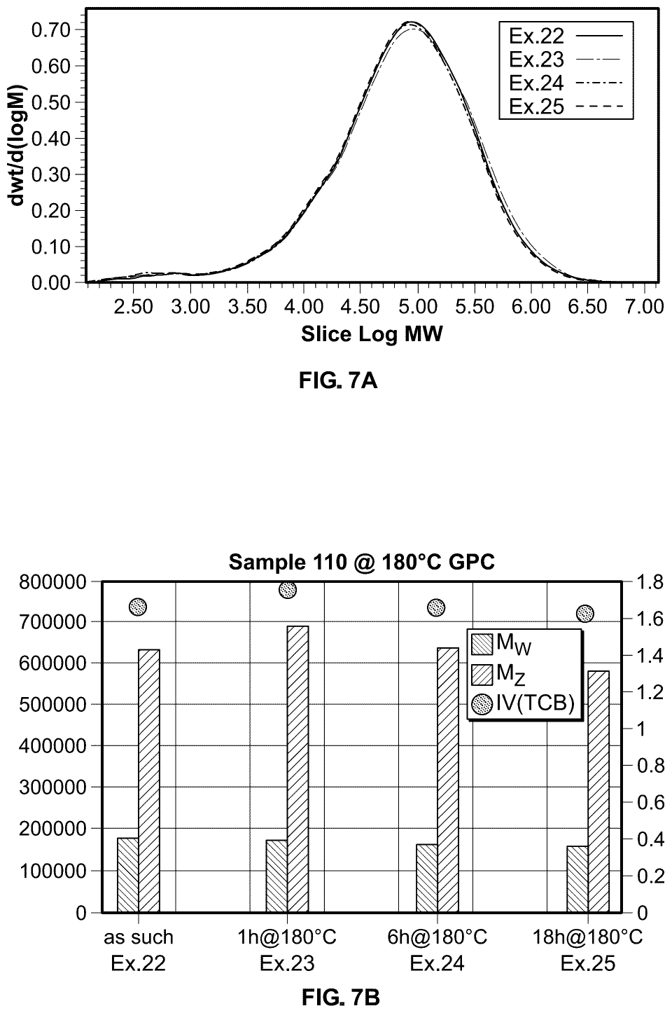

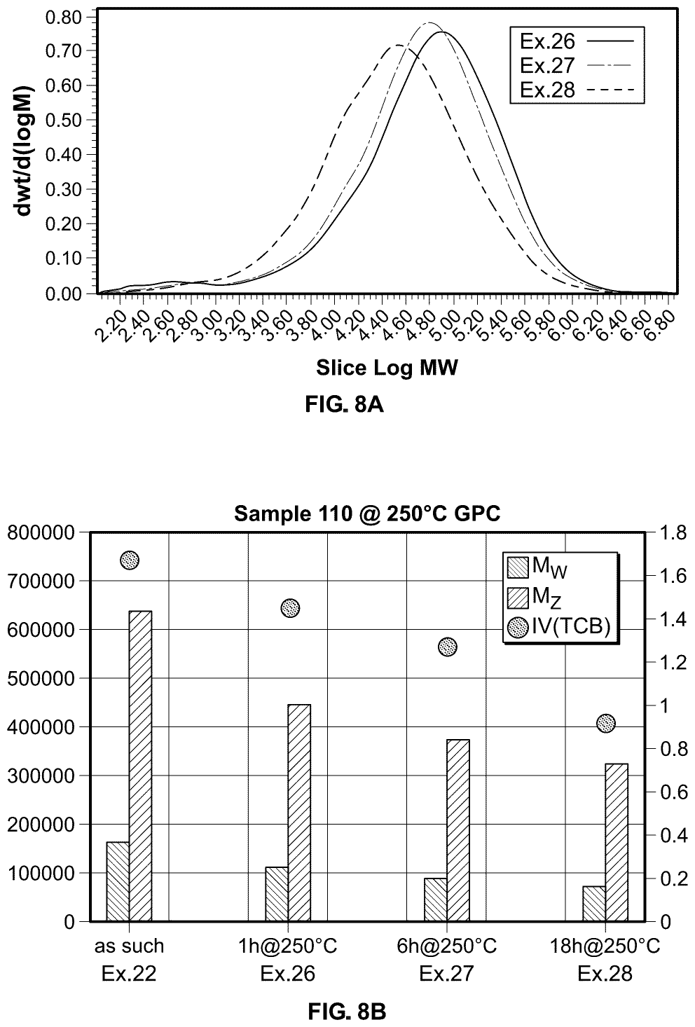

The results of Examples 22-28 are shown in FIG. 7A-FIG. 8B and summarized in Table 5 below.

TABLE 5

| | | | Weight | | | IV | | | | |
|---|---|---|---|---|---|---|---|---|---|---|
| | Time | Temp. | Loss | | | TCB | M_{w} bef./ | M_{z} bef./ | IV bef./ | Related |
| Ex. | (hrs) | (° C.) | (%) | M_{w} | M_{z} | (dl/g) | M_{w} aft. | M_{w} aft. | IV aft. | FIG. |
| 22 | — | — | — | 164,743 | 637,282 | 1.67 | 1.00 | 1.00 | 1.00 | 7A, 7B |
| 23 | 1 | 180 | 0.7 | 176,327 | 692,642 | 1.75 | 1.07 | 1.09 | 1.05 | |
| 24 | 6 | 180 | 1.7 | 163,312 | 637,865 | 1.65 | 0.99 | 1.00 | 0.99 | |
| 25 | 18 | 180 | 3.0 | 155,785 | 577,101 | 1.61 | 0.95 | 0.91 | 0.96 | |
| 26 | 1 | 250 | 0.7 | 109,969 | 446,339 | 1.45 | 0.67 | 0.70 | 0.87 | 8A, 8B |
| 27 | 6 | 250 | 1.7 | 91,645 | 374,463 | 1.27 | 0.56 | 0.59 | 0.76 | |
| 28 | 18 | 250 | 4.3 | 73,395 | 325,825 | 0.92 | 0.45 | 0.51 | 0.55 | |

Example 22 is untreated polymer recyclate PR2. Examples 23-25 show minimal reduction in M_{w}, M_{z}, and IV, even after 18 hours at 180° C. In contrast, Examples 26-28 show measurable reductions in all of M_{w}, M_{z}, and IV, after only 1 hour at 250° C. with continued reductions at 6 hours and 18 hours. Therefore, the method for pretreatment of polymer recyclates reduces the molecular weight of the polyolefin component as well as degrading the polar polymer component.

### Polymer Recyclate Pretreatment in Extruder

These experiments were performed using a Coperion co-rotating twin screw extruder (Coperion GmbH, Stuttgart, Germany) with a working in a temperature range of 220-250° C., with a dedicated vacuum system in the 1st zone of the extruder just before the self-cleaning filter. The extruder was equipped for nitrogen purge as needed in the range of from 50-200 L/hr in conjunction with polymer throughput of 50-100 kg/hr. Characterization data obtained by NMR showed that the PET content in polymer recyclate PR2 decreases after the extrusion step as shown in Table 6 below. This reduction in polar polymer content results in an improved polymerization feed stream and higher catalyst activity during the pyrolysis reaction. The reduced polar polymer component in the polymer recyclate will result in less hetero atoms (i.e., less catalyst poison) in the depolymerization process.

TABLE 6

| | | Ash | | | | |
|---|---|---|---|---|---|---|
| | | 800° C. | PE | PP | PO tot | PET |
| Ex. | PR2 | (wt %) | (wt %) | (wt %) | (wt %) | (wt %) |
| 29 | Before extrusion | 4.0 | 65.0 | 27.0 | 92.0 | 1.5 |
| 30 | After extrusion | 3.7 | 68.1 | 31.5 | 99.6 | 0.5 |

¹³C NMR spectra were acquired on a Bruker AV-600 spectrometer equipped with cryoprobe, operating at 160.91 MHz in the Fourier transform mode at 120° C. The peak of the CH₂ (for PE based materials) ethylene or P_{ββ} carbon (for PP based materials) (nomenclature according to C. J. Carman, R. A. Harrington and C. E. Wilkes, Macromolecules, 10, 3, 536 (1977)) were used as internal reference at 29.9 ppm or 21.8 ppm. The samples were dissolved in 1,1,2,2-tetrachloroethane-d2 at 120° C. with an 8% wt/v concentration. Each spectrum was acquired with a 900 pulse, 15 seconds of delay between pulses and CPD to remove 1H-13C coupling. 512 transients were stored in 32K data points using a spectral window of 9,000 Hz.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents and references cited herein, including testing procedures, publications, patents, journal articles, etc., are herein fully incorporated by reference for all jurisdictions in which such incorporation is permitted and to the extent such disclosure is consistent with the description of the present invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure of the present invention, processes, machines, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, methods, and/or steps.

What is claimed is:

1. A method for treating a polymer recyclate to produce an improved depolymerization feed, the method comprising:
a) adding a polymer recyclate to a reaction zone, wherein the polymer recyclate comprises a polyolefin component and a polar polymer component, wherein the polar polymer component comprises one or more heteroatom bonds;
b) purging the reaction zone with a gas to maintain an oxygen-free atmosphere in the reaction zone;
c) implementing reaction conditions in the reaction zone, the reaction conditions comprising a temperature and a time period:
i) sufficient to degrade at least a portion of the one or more heteroatom bonds to produce a degraded polar polymer component and a contaminant gas comprising hetero-containing compounds; and
ii) insufficient to decompose the polyolefin component; and
d) recovering the improved depolymerization feed from the reaction zone, wherein the improved depolymerization feed comprises the polyolefin component and the degraded polar polymer component.

2. The method of claim 1, wherein the contaminant gas is generated at a first rate (R1) and purging the reaction zone comprises:
a) adding an oxygen-free gas to the reaction zone at a second rate (R2); and
b) withdrawing a waste gas from the reaction zone at a third rate (R3);
wherein;
R1+R2=R3; and
R2/R1 is greater than or equal to 1.

3. The method of claim 1, wherein the polyolefin component comprises polyethylene and/or polypropylene in an amount greater than or equal to 50 wt %, based on the weight of the polymer recyclate.

4. The method of claim 1, wherein the polar polymer component comprises a cellulose, a polyvinyl chloride, a polyethylene terephthalate, a polystyrene, a polyamide, a polycarbonate, an ethylene vinyl alcohol, a rubber, or a combination thereof.

5. The method of claim 1, wherein the reaction conditions further comprise:
a) a temperature in the range of from 210° C. to 340° C.;
b) a pressure in the range of from −100 kPag to 70 MPag;
c) agitation ranging from stirring to mechanical shear to add specific energy of up to 0.6 kW·hr/kg to the polymer recyclate;
d) an inert atmosphere;
e) a reaction time in the range of from 1 minute to 20 hours; or
f) a combination thereof.

6. The method of claim 1, wherein the reaction conditions are implemented in an apparatus comprising mixing means, heating means, and/or gas injection and/or withdrawal means.

7. The method of claim 6, wherein the apparatus is an extruder.

8. The method of claim 1, further comprising adding a poison mitigation compound to the polymer recyclate.

9. The method of claim 8, wherein the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof.

10. The method of claim 8, wherein the poison mitigation compound is added in an amount less than or equal to 20 wt %, based on the total weight of the polymer recyclate and the poison mitigation compound.

11. The method of claim 1, wherein the reaction conditions are further sufficient to degrade the polyolefin component to form a degraded polyolefin component.

12. The method of claim 11, wherein:
a) the polyolefin component has a first weight average molecular weight (M_{w1});
b) the degraded polyolefin component has a second weight average molecular weight (M_{w2}); and
M_{w1}/M_{w2} is less than or equal to 0.9.

13. A process of depolymerizing a polyolefin recyclate, the process comprising:
a) treating a polymer recyclate according to the method of claim 1 to produce an improved depolymerization feed;
b) adding the improved depolymerization feed to a reaction zone in the absence of oxygen under depolymerization conditions sufficient to form a first vapor stream and first liquid stream comprising char;
c) adding the first vapor stream to a condensation zone wherein heat is removed to form a second vapor stream and a second liquid stream comprising one or more olefin monomers.

14. The process of claim 13, further comprising adding up to 20 wt % of a catalyst to the reaction zone, based on the total weight of the improved depolymerization feed and the catalyst.

15. The process of claim 14, wherein the catalyst comprises an acidic catalyst, a metal-based catalysts, a thermal catalyst, an activated carbon, or a combination thereof.

16. The process of claim 13, wherein step a) is performed in an extruder, and step b) is performed in a reactor vessel.

17. The process of claim 13, wherein step a) and step b) are performed in a reactor vessel.

18. The process of claim 13, wherein the improved depolymerization feed comprises polyethylene, polypropylene, or a combination thereof.

19. The process of claim 13, wherein the depolymerization conditions comprise a temperature in the range of from 400° C. to 600° C., a pressure in the range of from 1.0 barg (100 kPa) to 7.0 barg (700 kPa), or a combination thereof.

20. The process of claim 13, wherein conditions in the condensing zone comprise a temperature in the range of from 20° C. to 100° C., a pressure in the range of from 30 kPa to 200 kPa, or a combination thereof.
